(19) United States
(12) Patent Application Publication
(10) Pub. No.: US 2006/0078629 A1
(43) Pub. Date: Apr. 13, 2006

(54) Nutritional compositions and use thereof

(76) Inventor: Willem Jacob Serfontein, Pretoria (ZA)

Correspondence Address: Vani Moodley;Hahn and Moodley, Suite 180, 800 W El Camino Real, Mountain View, CA, 94040, US

(21) Appl. No.: 11/293,466

(22) Filed: Dec. 2, 2005

Related U.S. Application Data
(63) Continuation-in-part of application No. PCT/ZA04/00060, filed on Jun. 3, 2004.

(30) Foreign Application Priority Data
Jun. 4, 2003 (ZA) 2003/4360
Jul. 1, 2003 (ZA) 2003/5112
Aug. 28, 2003 (ZA) 2003/6713
Jan. 6, 2004 (ZA) 2004/0053

(51) Int. Cl. A61K 36/064; A61K 33/04; A61K 31/198

(52) U.S. Cl. 424/702; 424/195.16; 514/561

(57) ABSTRACT

A nutrient composition or combination of compositions for the treatment or prophylaxis of infections, in particular HIV/AIDS, and for the enhancement of immunity, based on selenium in synergistic combinations with biologically absorbable sources of glutathione, alkalinity enhancing components, a source of sulphur, an anti-mutagenic compound and for oral use, gastrointestinal absorption enhancers. Special uses relate to reducing risks of mother-to-child transmission and treating HIV-positive pregnant women. Preferred further ingredients include anti-inflammatory compounds and nutrients which control homocysteine.

### RELATED APPLICATIONS

This is a continuation in part of International Application PCT/ZA2004/000060, with an international filing date of Jun. 3, 2004.

### FIELD

The present invention relates to a nutrients supplementation composition or combination of compositions based on selenium and to a use thereof in antiviral treatment and/or prophylaxis and/or enhancement of the immune system in humans or animals.

### BACKGROUND

The HIV/AIDS pandemic is one of the greatest disasters in human history. It is estimated that by the year 2015, nearly 20%/o of the entire world population will be affected and 250 million people will have died of AIDS. According to recent reports, 70% of all young people in the Commonwealth Countries of Africa are now infected and in many African countries the overall population infection rates vary from 20 to 40% with a relentless upward trend. These figures highlight the seriousness of the problem, especially in Africa. They also underline the fact that measures adopted up till now to counter the relentless march of the epidemic, have been ineffective.

Anti-retroviral drugs such as AZT and others have some effect on the virus but the benefits are only temporary and they have had little or no impact on the progression of the disease world wide for reasons that are not difficult to understand. These include the horrendous side effects and especially the fact that they accelerate the evolution of new and resistant strains of the virus thus compounding rather than ameliorating the problem (1). Also the virus is never eradicated completely and the patient remains HIV+ for the rest of his/her life. This has two very important other practical disadvantages. Firstly, especially in the African setting, patients are given the impression that they are “cured” after drug treatment and this encourages them to practice unrestricted unprotected sex.

Also, since the patients are not cured (they remain HIV+), the ultimate effect of drug treatment is to increase the pool of infected people in the population thus aggravating the problem as a whole in the long term.

In addition, these drugs are very expensive and therefore unsuitable for use by the general population affected by the virus in many Third World countries.

There is therefore a pressing need for an approach that can be readily applied, particularly in the Third World situation.

The applicant is aware of an observation, made many years ago, that nutritional inadequacies are a major risk factor in the development of AIDS. However, the precise nature of the deficiencies involved remained obscure until about 10 years ago when leading international researchers, inter alia Dr E W Taylor of the University of Georgia and Prof H D Foster of the University of British Columbia, focussed their attention on the essential micro mineral selenium. The first indications that selenium may be involved came from epidemiological observations on disease prevalence in certain Sub-Saharan African countries. The AIDS incidence in these countries is generally very high ranging from approximately 20-40% and rapidly increasing in most of these countries (2, 3 and 5).

Senegal is however a noteworthy exception. There the incidence is of the order of 1% and virtually static (2). Although an educational program on AIDS has been in place in Senegal for some time (3), this only offers the illusion of protection since similar programs have been unsuccessfully implemented in other Sub-Saharan countries. Senegal is a desiccated Cretaceous and early Eocene sea bed rich in soil selenium compared to other African countries such as Botswana (4) and Uganda (5). By providing the much needed mineral selenium, the soil and food chain in Senegal creates a favourable environment for the human immune system. The food chain in that country provides, in addition to selenium, ample supplies of calcium and magnesium, the role of which has so far not been recognised. Apart from benefiting AIDS patients, this environment appears to have also provided protection against the multiple other infections to which people in these countries are subjected.

It also became known that selenium plays a fundamental role in the growth and mutations of many viruses, notably in the transformation of the normally harmless Coxsackie virus in the Keshan province of China where the virus had mutated to a much more virulent form in the presence of unusually low soil content of selenium. This caused an epidemic of cardiomyopathy in that province. Intervention by the Chinese government in the form of soil and food enrichment with selenium supplements brought the epidemic under control (6).

In addition, a similar relationship exists between low soil selenium levels and the incidence of AIDS in Western countries like the USA (7). This relationship is so strong and consistent that a recent World Atlas of soil selenium content in different countries of the world uses the HIV/AIDS incidence as a surrogate indicator of soil selenium content in those countries where analytical figures on soil selenium content are not available.

This strongly focussed attention on the possible link between the high selenium levels in Senegal and the low incidence of the disease in that country stimulated intense research during the last 10 years into this relationship, the most important findings of which—relevant to the present application—are as follows:

Long before Taylor's pioneering work, it had been known that AIDS patients had very low levels of selenium but it was assumed that this was just another side effect caused by the catabolic state induced by the virus. Taylor's work has own that the role of selenium in the AIDS patient goes much beyond this.
both the human host as well as the HIV virus require selenium for growth;
in the host selenium plays the role of an essential antioxidant which inter alia protects the host's immune system against the destructive effects of free radicals and viruses; thus reducing oxidative stress
the virus encodes the selenium containing enzyme glutathione peroxidase (GPx) thus competing with the host for available supplies of selenium;
in addition, the virus uses selenium as a growth regulator. When selenium supplies are adequate, the virus replicates slowly and the disease therefore progresses slowly or not at all. (This happens in Senegal where the incidence of the disease is more or less static in spite of the promiscuous sexual practices which do not differ from that in other African countries in the region. It also happens in the HIV positive patient who for long periods—even years—remains symptom free); and
When there is a deficiency of selenium, the virus interprets this as a signal to multiply (or otherwise face death due to selenium deficiency) and therefore spreads to neighbouring cells. This signifies rapid progression of the disease and therefore the development of clinical AIDS in the HIV positive patient. Taylor has proposed the existence of a regulatory protein, possibly even a “master switch” that switches on viral replication and which is switched on when there is a selenium deficiency.

This is consistent with the finding that AIDS patients with depleted selenium levels are 20 times more likely to die than those with adequate selenium levels (Chem Biol Interact 1994, 91: 181).
The selenium theory also explains why, after centuries of exposure to the simian strains of the virus, from which the human strain of HIV evolved in the Congo and other African countries, it is only during the last 20 years that AIDS has become a major clinical problem. This is due to the fact that the levels of selenium in the soil have been progressively depleted until critically low levels have now been reached. As will become apparent from the teachings of the present invention, the simultaneous depletion of many other vital minerals has been a contributory factor. Selenium is not required by plants and therefore never included in soil fertilisation programmes. Acid rain and over utilisation of agricultural soil are further contributory factors. More than just the presence of the virus is required before clinical AIDS develops.
The selenium status in the population determines who will become infected with the virus. This creates an opportunity to protect populations at large in situations where exposure cannot be prevented and this applies to most populations.

These observations suggest that administering selenium supplements to AIDS patients should be beneficial to them. It further suggests that by administering selenium supplements to populations at risk for the disease, the incidence of the disease should be reduced. However, the present invention is based on the concept that selenium supplementation alone is not enough to restore effective selenium blood levels and immunocompetence. Accordingly, the present invention teaches the administration of selenium in combination with other nutrients provided for in the present application to restore immunocompetence in the AIDS patient thus suppressing the well known opportunistic infections that are so typical of the condition and also to protect the immune system in African and other populations which are often plagued by a host of other infections. This concept was arrived at from a thorough analysis of a large number of clinical data collected from published as well as own clinical observations. These observations have on closer scrutiny led to the recognition of synergisms not previously known to exist.

### SUMMARY

Against the aforesaid background the present invention teaches a combination of factors which have to be applied in order to achieve maximum efficacy in the suppression of viral replication and/or mutation and/or for enhancing the immune system in humans or animals, thereby at the same time reducing the likelihood of viral infection or, where infection has already occurred, reducing the likelihood of acquired resistance.

The combinations taught by the invention act synergistically in that
a) the combination achieves benefits in excess of the sum total of benefits attainable by the individual factors;
b) the combination is effective in cases where the application of any one factor alone is ineffective or inadequate.

For example, the applicant has found that a combination of selenium dietary supplements and glutathione (GSH) system dietary supplements is synergistic.

Although in what follows the use of the invention in the important context of HIV/AIDS will be emphasized, it should be understood that the invention can have much wider applications in humans and animals, not only in anti-retroviral therapy and prophylaxis but also where other viruses are involved, e.g. ebola, coxsackie virus (Keshan disease), Hepatitis virus and immunology in general.

In its broadest sense, the invention provides nutrients supplementation compositions including biologically absorbable and acceptable selenium in combination with a source of glutathione or precursors thereof and one or more features designed to enhance the absorption/utilisation of selenium in cells of the body. The invention also provides uses of such compositions.

Thus, according to one aspect of the invention there is provided a nutrients supplementation composition or combination of compositions comprising:
a) one or more biologically absorbable and acceptable selenium compounds in synergistic combination with substances enhancing physiological selenium absorption and utilisation including
b) one or more biologically absorbable and acceptable sources of or precursors of glutathione (GSH) characterised in that
   a) is represented, in amounts to provide a daily dosage of not less than about 400 mcg Se, by
      a1) at least one selenium compound selected from methyl selenocysteine, selenomethionine, selenium yeast complex, selenium amino acid complex and/or
      a2) a plurality of selenium compounds; and
   there is also present
   c) a combination of biologically absorbable and acceptable blood and intracellular alkalinity enhancing components in amounts sufficient to enhance blood pH to above 7,45, including
      c1) one or more salts of calcium and/or magnesium and/or
      c2) a potassium salt of an organic acid and
      c3) one or more salts of cesium and/or rubidium and/or
      c4) a lithium salt.

In addition, the composition or group of compositions may comprise the following combination of features:—
d) a biologically absorbable and acceptable source of sulphur; and
e) one or more biologically absorbable and acceptable anti-mutagenic compounds; and
f) in the event of compositions for oral administration, one or more gastrointestinal absorption enhancers for selenium; and
g) one or more gastrointestinal protectors.

More specifically, the selenium compound or compounds is/are selected from the group consisting of selenocysteine, selenomethionine, methylselenocysteine (MSC), methylselenomethionine, alkali metal selenites, selenium yeast complex, proteins incorporating selenium, selenium analogues of sulphur amino acids, amino acid complexes of selenium, the substance known in the trade as “selenium amino acid chelate, selenium complexed with coral calcium, analogues and derivatives of the aforegoing and combinations of a plurality of the aforegoing. Generally speaking, organic compounds of selenium are preferred.

Whereas the usual doses of elemental selenium supplementation are in the range of 100-200 μg daily, at least 400 μg are preferably required in the case of many AIDS patients. This is due to the fact that in many HIV/AIDS patients, selenium absorption is less efficient than in controls. As long as dosage levels do not exceed 800-1000 μg per day, toxicity is non-existent.

The commonly used sources of organic selenium are methylselenocysteine, methylselenomethionine, selenium yeast and “selenium amino acid chelate”. The latter is available under that name in the trade but it is a misnomer because selenium does not form chelates. Applicant has found that for the purposes of the present application, the first two are both suitable compounds. Applicant has also found methylselenocysteine (MSC) to be the more biologically available compound of these two. MSC is therefore the preferred compound, especially for use in the treatment of clinical AIDS with intravenous formulations according to the invention. MSC is an essential component of the enzyme glutathione peroxidase, which as previously explained, is known to protect cells against oxidative damage (Alt Complem Therap 2000, 6:342) and, for the purposes of the present invention, also against viruses and specifically against the HIV.

It is known that MSC is one of the most effective forms of selenium for the prevention of cancer (Nutrition and Cancer 2001, 40:12).

The invention teaches that, with regard to the activity of selenium compounds, there is a parallelism between cancer prevention and AIDS prevention and treatment.

It is of utmost importance for selenium to be effective to take care that the glutathione (GSH) is kept effective in patients. This may be achieved by a GSH system dietary supplement more particularly a GSH blood level enhancing supplement and/or a GSH system efficiency enhancing supplement. In some cases GSH itself may be administered. However, the ability to absorb GSH itself is compromised in many patients for which reason precursors of GSH are generally preferred. Therefore, preferably (in b) above) the precursor(s) of glutathione is/are selected from the group consisting of acylcysteines, N-acetylcysteine (NAC), N-propionyl cysteine, N-butyl cysteine, Lipoic acid, methyl sulphonyl methane (MSM), analogues and derivatives of the aforegoing and combinations of a plurality of the aforegoing.

One objective of the aforegoing feature will be modifying the GSH:GSSG to the desired level and ratio, as explained below. The N-propionyl and N-butyl derivatives may have advantages over the more widely available N-acetyl derivatives but the latter may be the preferred compound due to cost considerations.

The applicant has found that, in the patient, the existence of inadequate levels of GSH is reflected in a decreased ratio of oxidised GSH (GSSG) to reduced GSH.

These relationships are illustrated in the following scheme in which
GSH=reduced glutathione;
GSSG=oxidised glutathione; and
GPx=glutathione peroxidase.

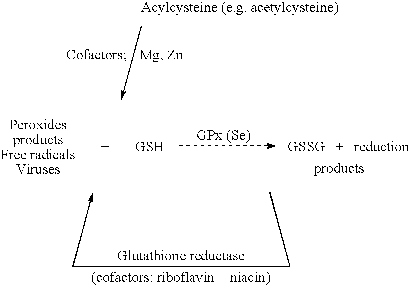

Accumulation of GSSG indicates inadequate glutathione generating capacity in the cells. Normally this ratio is substantially greater than 100:1, e.g. in most cells more than 500:1 in the healthy patient with a fully functional glutathione system. Applicant has found reduced ratios in many AIDS patients. In some this ratio may be as low as 50:1 or even much lower. One aspect of the invention therefore provides for conditions that will ensure adequate levels of glutathione. The conditions must also be such that glutathione is maintained predominantly in its reduced state. Both of these conditions can be attained by providing adequate quantities of glutathione precursors (e.g. cysteine) in addition to the cofactors required for the conversion of the cofactors necessary for maintaining GSH in the reduced state (Mg, Zn). In addition, adequate levels of cofactors required to maintain glutathione in the reduced state (e.g. riboflavin and niacin) must be present simultaneously.

The invention teaches that selenium can only have an effect through the selenium containing enzyme GPx which in turn can only exert its clinical effects if adequate quantities of GSH are present and if relatively alkaline conditions exist in the body (e.g. blood pH above 7,45 and urinary and saliva pH above 6,2). The importance of alkaline conditions in the blood (to be distinguished from the mere presence of elements such as Ca and Mg) has not in the past been paid attention to. This is the significance of c) above.

Preferably, the blood alkalinity enhancing compound(s) is/are selected from the group consisting of alkalinity enhancing calcium, magnesium and potassium compounds, lactates, citrates, tartrates, malates or other fruit acid salts of the aforegoing, calcium carbonate, magnesium carbonate, basic magnesium carbonate, magnesium oxide, dolomite, coral calcium, analogues and derivatives of the aforegoing and combinations of a plurality of the aforegoing.

Blood pH (acidity) in humans is determined by the diet (and ultimately by the minerals in the soil on which plants used in the diet grow). Blood pH values normally range from 7,35 (extremely acidic) to 7,45. Relatively alkaline blood pH values are anabolic (tissue building) and therefore health promoting while more acidic conditions are associated with catabolism and therefore unhealthy, especially in the AIDS patient. Many normal metabolic processes are associated with acid production which is then balanced by the blood buffer systems and minerals such as calcium and magnesium. When there is a deficiency of these alkalinising minerals, such a pH correction becomes ineffective with the result that acidosis develops.

Applicant has found that these considerations are particularly relevant in the AIDS patient in whom a strong catabolic tendency with associated tissue loss is a prominent feature. In addition, the immune system functions best under relatively alkaline conditions (pH above 7,45).

Applicant has found a widespread tendency towards acidosis in AIDS patients (blood pH below 7,4 and urinary and saliva pH values lower than 5,5).

Such acidotic conditions affect the immune system very unfavourably and are therefore of special significance in the AIDS patient. Moreover, besides elevating blood pH, the substances according to category c) also promote alkaline conditions in the gut. The invention teaches that, contrary to what one might expect, the use of acidity control buffers of category c) not only supports the immune system, but also improves the absorption of selenium through the gastro-intestinal tract which is frequently a problem in the AIDS patient.

The preferred dosages for alkalinising substances will be apparent from the examples. Particularly in the case of patients having a shortage of cesium and/or rubidium, it is preferred to include cesium and/or rubidium compounds in combination with a source of calcium for enhancing pH levels and, in particular, also intracellular pH levels. The invention teaches that intracellular alkalinity is of particular importance.

In addition, it has been found that lithium plays an important role in a manner which appears to go beyond mere pH control. Accordingly, the composition or combination of compositions to be administered preferably includes a source of lithium, more particularly in a form suitable for lithium to be carried to cellular membranes. Preferably, the source of lithium is selected from organic lithium salts of the group consisting of lithium orotate, lithium aspartate, lithium salts of fatty acids, polyunsaturated fatty acids, those that occur in phospholipids in cellular membranes; DHA, EPA, [symbol] -linolenic acid, palmitic acid, stearic acid and other acids that occur in biological membranes or from lithium selenite or lithium selenate and combinations of a plurality of the aforegoing.

Lithium is to be administered to AIDS patients in very low dosages of 5-20 mg daily. Careful patient monitoring is necessary because even at such low concentrations of lithium, some patients may experience slight toxicity side effects.

For that reason, it is preferred to employ even considerably lower “ultra low” dosages of lithium, of 20-500 mcg lithium. At such low dosages (which are new and inventive per se), it is possible to employ lithium in long-term therapy as a supplement in the treatment program of AIDS patients, especially those with AIDS defining complications of the disease which are related to immune and nervous disorders.

The lithium salts can be conveniently prepared by mixing the calculated amount of lithium carbonate or lithium hydroxide with the organic acid in pure form or in a suitable hydrophilic/hydrophobic solvent mix. In the slow reaction that follows, the organic acid is partly converted to the corresponding lithium salt.

The reaction is usually carried out in the presence of a 10 fold excess of the acid in a 50% ethanol medium and the resulting mixture is then sprayed on to the rest of the ingredients in the supplement corresponding to one daily dose.

The invention further teaches that lithium selenite (or lithium selenate) are also suitable as selenium sources in supplements. Lithium selenite is the preferred form (LiSeO3. H₂O). It has the additional advantage of also supplying lithium and selenium in the same dosage. Thus a daily dose of 385 mcg of lithium selenite will supply 200 mcg of selenium and 17,6 mcg of lithium.

Glutathione is a tripeptide which consists of the three amino acids glutamic acid, cysteine and glycine. Of these, the sulphur containing amino acid cysteine is the part of the molecule where its principal activity as sulfhydryl compound is situated. As indicated in the above scheme of the glutathione system, it can undergo reversible oxidation-reduction thereby acting as an anti-oxidant. In addition, glutathione has other beneficial effects in the AIDS patient.

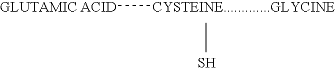

The structural formula of reduced glutathione illustrates the importance of sulphur in the structure of glutathione.

Applicant has recognised that, from a therapeutic point of view, an optimally functioning glutathione system is a critical factor in the treatment and prevention of AIDS and indeed a prerequisite for any form of treatment including drug treatment. Selenium exerts effects through the glutathione system but it is equally true to say that without selenium, the glutathione system cannot function. Patients with low levels of glutathione have been shown to have a much reduced life expectancy.

One method of assessing the functional activity of the glutathione system is to measure the ratio of reduced glutathione: oxidised glutathione. This ratio is different in different cells but in most cells this ratio is higher than 500:1.

Cysteine, which carries the sulphur atom in the glutathione molecule, derives its sulphur from homocysteine:
Homocysteine+serine→cysteine+ketoglutarate

Homocysteine is derived from methionine in methylation reactions and methionine in turn is degraded into propionyl-ScoA whence the sulphur may be further degraded to simple sulphur compounds which make up the body's sulphur pool.

Many of these reactions are reversible under certain circumstances so that ultimately the sulphur in glutathione is drawn from the body's pool of sulphur compounds some of which are derived from the diet.

Therefore the body's pool of sulphur compounds is of great significance in maintaining adequate levels of glutathione.

The status of the body's pool of sulphur compounds may be judged from the daily excretion of sulphur compounds in the urine.

Normally, the whole blood contains 3,84-5,06 mg of sulphur per 100 ml (excluding sulphur present in proteins (Z Klin Med 1940, 137:467). Whole body sulphur content has been reported as 0,196 g/100 g while the glutathione content of plasma from normal humans is 0,91±0.24 micromoles/l. The relationship between glutathione levels and AIDS has been explored by the Herzenbergs in New York. They showed that the patients' glutathione levels determine the length of time that they will survive. Other studies have also shown that high glutathione levels significantly increase survival times in people with AIDS and that they may be correlated with immune cell (CD4) subcell counts (AIDS 1992, 47: 1021).

Twenty four hour urinary excretion (total sulphur) in normal adults has been reported to be 2,0-3,4 g.

In contrast to normal people, applicant has found that in AIDS patients (depending on the stage of the disease) urinary daily sulphur loss may be as high as 8-12 g which leads to a substantial sulphur loss which cumulatively over time will have severe negative health effects and in particular to adversely affect the body's sulphur pool and therefore the body's capacity to maintain adequate levels of glutathione. Although this massive loss of sulphur may be partly related to the general debilitating condition of many AIDS patients similar to the loss of other nutrients such as nitrogen, it was not previously recognised that the consequences of the loss of sulphur has other consequences apart from general tissue loss, since it directly affects the body's ability to fight the HIV virus.

All the important functions of GSH outlined above are dependent on the presence of adequate levels of the selenium containing enzyme glutathione peroxidase (GPx) and therefore of adequate levels of selenium.

Restoring the body's sulphur pool is therefore one aspect of the present invention. This is best achieved by administering suitable biologically available sulphur compounds such as methyl sulphonylmethane (MSM).

Normally sulphur is obtained from the foods (plants, animals) that we eat. Sulphur is obtained from the plants that we eat which in turn obtain their sulphur from the soil on which the plants grow. Ultimately therefore the sulphur status of humans (like the selenium status) depends on the content of these minerals in the soil.

It is interesting to note that there is some parallelism between the contents of these minerals in the soil of different regions and different countries which appears to have a bearing on the geographical incidence of AIDS.

The declining soil content of these and other minerals are closely linked to destructive agricultural practices coupled to selective fertilisation programs which do not include sulphur and selenium as well as other environmental factors such as acid rain. Many health consequences of a sulphur deficiency in humans have been described. These include gastrointestinal problems and a poorly functioning immune system, both of which are of special significance in the AIDS patient. Both of these problems exist in the AIDS patient with a selenium deficiency and clearly, a co-existing sulphur deficiency will aggravate these conditions.

Thus, according to one aspect of the invention, the massive sulphur loss that is common in AIDS patients, is corrected by means of a suitable supplementation program with sulphur.

Methyl sulphonyl methane (MSM) is an excellent supplemental source of sulphur in contrast to many other inorganic forms of sulphur that may be toxic.

It is part of nature's sulphur cycle and occurs naturally in tissues and fluids of plants and animals including humans. Applicant has recognised that MSM is non toxic, has no side effects and is a completely safe way of supplementing the body of AIDS patients with sulphur that is needed for the body as a whole to function best and specifically to correct the extensive loss of sulphur that occurs in these patients.

Thus, according to the invention, in c) above the organic source of sulphur is selected from the group consisting of cysteine, methyl sulphonylmethane (MSM), sulphur amino acids, cystine, lanthionine, sulphur containing polypeptides, alkali metal thiosulphates, preferably sodium thiosulphate, analogues and derivatives of the aforegoing and combinations of a plurality of the aforegoing. Sodium thiosulphate (Na₂ S₂ O₃) is a source of biological sulphur that, according to the invention, is particularly useful to boost the body's sulphur reserves. It is non-toxic and contains readily available sulphur.

Reverse transcriptase is the name given to the RNA directed DNA polymerase by means of which the retroviruses translate their RNA based genetic “message” into DNA code. The life cycle of a typical retrovirus such as HIV starts with the infecting virions (complete viruses) binding to specific cell receptors on the surface of the host cell and entering the host cell. Thereafter transcription of the RNA message occurs only after the virus has entered (integrated with) the host cell DNA. This integration is an obligatory step in the life cycle of retroviruses. A similar process occurs when chemical carcinogens attack the host cell's DNA to produce new aberrant (e.g. cancerous) cell lines. It is at this level that DNA protectors such a chlorophyllin exert their protective effect (anti-mutagenic effect) (Mutation Res 1997, 376:97). Before such integration can take place, a carcinogen or virus must be able to form adducts with DNA (Env Mol Mutations 1996, 27:211). When a high enough percentage of such DNA adducts form along critical gene segments, normal cells are transformed into aberrant cells which may either become cancerous or, in the case of viral attack, be transformed into cells that reproduce viruses. Chlorophyllin has the property of trapping chemical carcinogens by reacting with their “back bone” thus making it impossible for them to form adducts with DNA thus preventing them from initiating the process of carcinogenesis (Env Mol Mutation Res 1997, 388:79).

The present invention teaches that the same process happens in the case of viruses thus preventing or suppressing their fusion with the host cell (e.g. immune cell DNA).

Comparative studies have shown that although there are many chemical compounds in natural products (e.g. teas) that have similar properties, chlorophyllin is by far the most potent antimutagen available. However, other antimutagens may be used in addition or as an alternative, in particular substances which also exercise an antimutagenic effect in the context of certain cancers. Preferably, in the context of item e) above the anti-mutagenic compound(s) is/are selected from the group consisting of chlorophyllin, indole-3-carbinol (I3C), folic acid, niacin, niacinamide, analogues and derivatives of the aforegoing and combinations of a plurality of the aforegoing.

The nutritional formulation may also include amino acid supplements.

The amino acid supplements may include cysteine, glutamine and tryptophan.

The nutritional formulation may also include a compound which increases absorption of certain compounds.

The applicant has found that glutamine has many beneficial effects in the AIDS patient. In addition to promoting the synthesis of glutathione (which is of prime importance in the AIDS patient), it maintains the structural integrity of the intestines which is frequently compromised in the AIDS patient to the extent that it has been referred to as the “intestinal permeability factor”. Its main function is to restore the health of the mucous membranes in various segments of the gastro-intestinal canal. In general it lessens the inflammation in the gut that is frequently present in AIDS patients.

The applicant is further aware that piperine is a mild irritant which has been shown to increase the absorption of certain compounds.

The compound which increases absorption of certain compounds may be selected from L-glutamine and piperine.

Thus, a preferred meaning of f) above is one, wherein a gastrointestinal protector is selected from L-glutamine, analogues and derivatives of the aforegoing and combinations of a plurality of the aforegoing.

Likewise, a preferred meaning of g) above is one, wherein a gastrointestinal absorption enhancer for selenium is selected from piperine, L-glutamine, analogues and derivatives of the aforegoing and combinations of a plurality of the aforegoing.

Preferably, a) is combined with two or more of b) to g).

Preferably, a) is combined with b) and c).

Advantageously, a) is combined with b) and c) and at least one of d) and e).

However, most preferably, all categories a) to g) are represented.

It should be understood that in certain embodiments at least one compound performs the function of more than one of the categories of a) to g).

The invention further teaches that, in addition to the categories a) to g) in order to enhance the effect thereof, the following should preferably be provided:—

Firstly, because the HIV/AIDS is frequently aggravated by a depletion of specific micronutrients, the composition or combination of compositions should preferably contain micronutrients represented by mineral supplements selected from the group consisting of magnesium, manganese and zinc, and/or vitamins selected from the group consisting of vitamins A, B2, B3, B6, C, carotenoids and E and combinations of a plurality of the aforegoing.

Secondly, because inflammatory conditions have been found to play an important role in the transmission and dissemination as well as the symptoms of HIV/AIDS, the composition or combination of compositions according to the invention preferably contains one or more substances for modulating cytokine activity, selected from the group consisting of substances suppressing Tumor Necrosis Factor-alpha (TNFα) and interleukins 1 and 6, nettle leaf extract, pentoxifilline, curcumine, antioxidants, NAC, α-lipoic acid, analogues and derivatives of the aforegoing and combinations of a plurality of the aforegoing. The aforegoing substances are selected to avoid upsetting the balance of pro- and anti-inflammatory forces in the body. For that reason certain traditional anti-inflammatory drugs (e.g. salicylates and COX-inhibitors) are less preferred.

The administration of the substances for modulating cytokine activity is preferably modulated and adjusted to maintain levels of inflammatory agents in AIDS patients not to exceed the following maximum levels:

| PRO-INFLAMMATORY CYTOKINE | MAXIMUM LEVELS |
|---|---|
| Tumor necrosis factor α (TNFα) | Below 10 pg/ml |
| Interleukin-6 (IL-6) | Below 12 pg/ml |
| Interleukin-1β (IL-1β) | Below 15 pg/ml |
| Leukotriene B4 (LTB4) | Below 200 pg/ml |
| C-reactive protein (CRP | Below 1.5 mg/L |

Thirdly, because it has now been found that homocysteine levels increase to harmful levels in AIDS patients as the disease progresses, the invention teaches the inclusion of a substance or combination of substances for reducing homocysteine levels in blood, in particular trimethylglycine (TMG). The latter is recommended because the inventor has found that supplementation with vitamins B6, B12 and folic acid is frequently not sufficient to reduce homocysteine to desirable levels (not above 8,0 mmole/L) in AIDS patients. In addition to the normal health risks referred to above associated with increased homocysteine levels, homocysteine creates additional problems in the HIV positive patient as a result of its damaging effect on biological membranes in general. Damage to delicate barrier membranes in these tissues enhances the likelihood of virus transmission and promotes viral replication in the AIDS patient.

Fourthly, having regard to the importance of the selenium-containing glutathione peroxidase enzyme system in the context of the invention and the observation that certain amino acids of that system become depleted in viral infections, and in particular HIV/AIDS, the invention teaches that the composition or combination of compositions should preferably include replacement nutrients for the amino acids contained in the selenium-containing glutathione peroxidase enzyme system, selected from the group consisting of cysteine, glutamine, tryptophan, precursors, derivatives and analogues of the aforegoing and combinations of a plurality of these.

A further important factor relates to the gastrointestinal health in the AIDS patient. There is an extremely important relationship between the HIV virus, the AIDS wasting syndrome and the health of the gastrointestinal system, inflammatory conditions in the gut and TNFα levels in the gut which has been poorly appreciated in the past and which is not reflected in current treatment schedules. One of the fundamental guidelines on which the present invention is based, is that the recovery of the AIDS patient is greatly hampered in the presence of intestinal dysbiosis.

The present invention recognises that supplementation with probiotics may be less effective unless these are given simultaneously in conjunction with certain other provisions such as selenium supplements and correction of systemic acidosis as herein specified.

In addition, intestinal dysbiosis is associated with reduced nutrient absorption including the absorption of selenium. Thus by administering selenium in combination with an appropriate mix and dose of probiotic organisms such as lactobacilli and bifido bacilli, selenium absorption may be substantially improved. There is a subpopulation of AIDS patients that absorb selenium very poorly. We have found that in a large segment of this group, the problem is caused by intestinal dysbiosis.

Important exchanges occur between the luminal contents, the intestinal mucosa (to which normal beneficial gut micro organisms adhere) and the gut associated lymphoid tissue (GALT) in such a manner that this mechanism becomes an important determinant of overall systemic immunity. In fact, the GALT harbours several times more immune cell elements than all the other lymphoid tissues in the body combined (Surgery 1988, 104: 917). The system is adversely affected by inflammatory conditions in the gut wall as a result of which TNFα levels are raised which in turn promote replication of HIV virus via the NF-kβ system. Thus, TNFα is responsible for an early and essential step in virus replication. According to the present invention, this process may be controlled in three fundamental ways:
by controlling TNFα levels as discussed above
by providing a suitable mix or normal probiotic organisms to suppress luminal inflammation with special provision for those acid sensitive organisms specifically compromised in the AIDS patient and by the simultaneous provision of selenium which has an inverse relationship with pro-inflammatory cytokines such as TNFα.
By providing selenium to further control virus replication and support the action of the probiotics.

In infants exposed to the HIV, it is important to provide the correct type of micro organisms e.g. lyophilised cultures containing inter alia lactobacillus infantis. In general, probiotics may be administered (preferably as tablets, capsules, capslets) or administered to patients as an integral part of a multicomponent anti-AIDS supplement (see examples) or as part of a multivitamin/mineral formulation or they may be separately formulated as a probiotic supplement for AIDS patients. In this context the invention further teaches the inclusion of a source of probiotics.

Finally, according to a specific, important aspect of the present invention, the composition or combination of compositions and the various aforegoing teachings are applied to the treatment of HIV-positive pregnant women in order to reduce their HI-viral loads, and/or strengthen their immune system and/or mitigate or delay the onset of AIDS symptoms and/or reduce the risk of and/or counteract the effect on the foetus and neonate of mother-to-child transmission (MTCT) prior to, during or after parturition and/or to the treatment of newborn infants of such women.

Apart from what has already been taught further above, the composition or combination of compositions preferably includes a multivitamin/mineral formulation, specifically formulated to counteract deficiencies characteristic of HIV-positive pregnant women and/or of HIV-positive mothers of neonates and their infants.

According to one embodiment the composition or combination of compositions is or includes a parenteral selenium formula and is used during the third trimester and especially during the peripartum period of HIV-positive pregnant women.

According to another embodiment, the composition or combination of compositions is or includes a parenteral selenium formula and is used in new born infants of HIV-positive mothers, especially during the first 3 months after birth.

In the context of preserving the health status of HIV-positive pregnant women and of avoiding mother-to-child transmission (MTCT) of the HIV, it is of particular importance to include a source or sources of glutathione and/or substance(s) for enhancing the activity of the glutathione redox system in combination with an alkalinising substance or substances. It is preferred that the alkalinising substance or substances is/are formulated to achieve daily dosage ranges in accordance with the following:

| | Mother (mg) | | Infant (mg per kg body wt) | |
|---|---|---|---|---|
| COMPOUND | RANGE | PREFERRED | RANGE | PREFERRED |
| Calcium (as CaCO3) | 100-1500 | 300-800 | 1.5-25 | 5-15 |
| Magnesium (as MgCO3) | 20-500 | 100-300 | 0.3-7.5 | 1.5-5.0 |
| Basic Mg (as basic MgCO₃) | 20-500 | 100-300 | 0.3-7.5 | 1.5-5.0 |
| Mg (as citrate basic) | 20-500 | 100-300 | 0.3-7.5 | 1.5-5.0 |
| Ca as citrate (basic) | 100-1500 | 300-800 | 1.5-25 | 5.0-15 |
| Ca as bisglyuriate | 100-1200 | 300-600 | 1.5-20 | 5.0-10 |
| Potassium citrate | 500-3000 | 500-1500 | 7.5-45 | 7.5-22.5 |

The above dosage ranges are also the preferred ranges for patients other than pregnant women and infants.

The present aspect of the invention is based on the concept that additional considerations and factors apply to the treatment of HIV-positive pregnant women and in the context of mitigating the rate and effects of MTCT to their foetuses and newborns.

The rate of transmission of MTCT ranges from 15-30% (average 25%) which is unexpectedly low. Transmission occurs in utero (transplacental transmission), during labour and delivery and post partum through breast milk. Most of the transmission occurs in late pregnancy and during labour. Some factors that increase transmission are: maternal viral load, clinical, immunological and nutritional status of the mother, presence of other factors that may harm the immune system (e.g. drugs, foreign chemicals) and rupture of membranes during delivery.

Based on a careful analysis of observations and data, the invention teaches that nutritional factors are at least as important as viral load in vertical transmission from mother to child. They may reduce transmission by affecting several maternal and foetal risk factors for transmission such as immune status in both mother and child, effects of rate of viral progression, level of viral shedding in genital secretions and viral secretion in breast milk. Other factors in which nutrition play a role include reduction of low birth weight and maintenance of gastro-intestinal integrity in both mother and child. The virus has been shown to be present in most of the fluid secretions of the body (blood, serum, lymph, breast milk, semen, vaginal secretions etc.). During gestation and especially during the pre-partum, intra-partum and post-partum phases, there is ample exposure of the foetus to such secretions and on the basis of this, one would expect a transmission rate of near 100%. Yet under practical circumstances, the average transmission rate is only 20-30% and in some studies it has been reported to be as low as 10%. This leads to the concept of lowering the transmission rate by applying the inventive principles described in the aforegoing to
reduce the number of viruses physically transmitted;
modify the composition of the tissues and fluids into which the virus is transferred,
   thereby reducing the risk of infection of the foetus or infant and, should infection occur, reducing the severity thereof.

During pregnancy the selenium levels and other critical protective blood ingredients are transferred from the mother-to-be to the foetus, thereby improving the resistance of the foetus to HIV infection, whilst decreasing these levels in the mother-to-be. This transfer of critical nutrients from the mother to the infant and her resultant low selenium levels continue during the first few months post partum and are responsible for the increased AIDS mortality seen in lactating mothers compared to bottle feeding mothers. Our observations show high selenium levels in colostrum. The infant is protected thereby at the expense of the mother's own selenium supply.

The virus responds to selenium levels in one of two ways. When levels are low, the virus responds by increased proliferation resulting in rapid progression to full blown AIDS in the HIV positive patient. When levels are high, viral proliferation is repressed even to such an extent that the virus may remain dormant for long periods

Thus by increasing selenium levels in the infant at her own expense, the mother ensures that the infant is protected against viral proliferation whilst at the same time increasing her own risk of developing clinical AIDS.

The extent to which this protective effect operates obviously depends on the level of the mother's selenium reserves and when these are low, the infant is less well protected as happens in the case of the 20-25% of infants that are born infected.

The following table summarises the preferred daily administration ranges according to which the compositions in accordance with the invention are to be formulated.

| | | | INFANT | |
|---|---|---|---|---|
| | ADULT/MOTHER | | per kg | |
| COMPOUND | RANGE | PREFERRED | RANGE | PREFERRED |
| SELENIUM AS (mcg) | | | | |
| methyl selenocysteine (mg) | 50-1000 | 100-500 | 1.0-15 | 2-7 |
| selenomethionine (mg) | 50-1000 | 100-500 | 1.0-15 | 2-7 |
| sodium selenite (mg) | 50-600 | 100-400 | 1.0-8.0 | 2-5 |
| yeast complex (mg) | 50-1000 | 100-500 | 1.0-15 | 2-7 |
| total | 50-1200 | 400-800 | 1-17 | 5-12 |
| Folic acid (mg) | 0.02-10 | 1-5.0 | 0.0003-0.14 | 0.014-0.07 |
| Chlorophyllin (mg) | 50-1000 | 100-500 | 1-15 | 2-7 |
| L-tryptophan (mg) | 50-3000 | 500-1500 | 1-40 | 10-20 |
| L-glutamine (mg) | 50-30000 | 500-5000 | 1-400 | 10-70 |
| L-cysteine (mg) | 50-2000 | 300-600 | 1-28 | 4-8 |
| Niacin (niacinamide((mg) | 5-500 | 20-100 | 0.07-7 | 0.3-1.5 |
| N-acetyl cysteine (NAC) (mg) | 50-3000 | 300-1000 | 1-40 | 4-14 |
| Calcium (different sources) (mg) | 100-1500 | 300-800 | 1.4-20 | 4-11 |
| Magnesium (diff. sources) (mg) | 20-500 | 100-300 | 0.3-7 | 1.4-5 |
| Vitamin E (TE) | 10-1000 | 20-100 | 0.14-14 | 0.3-1.4 |
| Methyl sulfonylmethane (mg) | 50-5000 | 100-1000 | 1-70 | 2-14 |
| Riboflavin (mg) | 2-100 | 5-20 | 0.03-1.4 | 0.07-0.3 |
| Zinc (diff. sources) (mg) | 5-60 | 10-40 | 0.07-0.9 | 0.14-0.6 |
| Potassium citrate (mg) | 500-10000 | 700-2000 | 7-140 | 10-28 |
| Sod. Thiosulfate (mg) | 500-5000 | 700-2000 | 7-70 | 10-28 |
| Quercitin (mg) | 60-1500 | 600-1200 | 0.9-21 | 9-17 |
| Vitamin A (IU) | 5000-25000 | 10000-20000 | 70-350 | 140-280 |
| Ascorbic acid (mg) | 50-5000 | 200-1500 | 1-70 | 3-20 |
| Indole-3 carbinol (mg) | 50-1000 | 100-400 | 1-14 | 1.4-7 |
| β-Carotene (mg) | 5-100 | 10-50 | 0.1-1.4 | 0.2-1.0 |
| Pyridoxine (mg) | 5-30 | 5-30 | 0.04-1.4 | 0.07-0.42 |
| Vitamin C (mg) | 50-3000 | 100-2000 | 1.0-40 | 1.4-28 |
| Vitamin B12 (mcg) | 1-100 | 5-50 | 0.014-1.4 | 0.07-0.7 |
| Betaine (TMG) (mg) | 100-3000 | 500-1500 | 1.4-20 | 0.7-10 |
| α-lipoic acid (mg) | 100-1200 | 500-900 | 1.4-10 | 5.0-12 |
| Ca-d-pantothenate (mg) | 10-1000 | 100-600 | 0.1-10 | 1.0-6.0 |
| L-Carnitine (mg) | 100-3000 | 500-2000 | 1.4-30 | 7-20 |
| Pentoxyfillin (mg) | 100-800 | 400-600 | 1.4-12 | 5-8 |
| Nettle leaf extract (mg) | 400-1200 | 700-900 | 5.7-17 | 10-13 |
| Cesium chloride | 50-5000 | 100-2000 | 0.7-70 | 1.5-30 |
| Humic acid | 10-500 | 50-200 | 0.1-7 | 0.7-3 |
| Fulvic acid | 5-500 | 30-200 | 0.07-7 | 0.3-3 |
| Probiotic supplement | | | | |
| 1-5 × 10⁹ organisms | 100-1200 | 400-800 | 1.4-18 | 5.7-12 |
| per dose (mg) | | | | |
| Lithium selenite (mg) | 50-1000 | 100-400 | 0.7-15 | 1.4-6 |
| (LiSeO₃H₂O) | | | | |
| Lithium selenate (mg) | 50-1000 | 100-400 | 0.7-15 | 1.4-6 |
| (LiSeO₄.H₂O) | | | | |
| Li as PUFA complex (mg) | 10-500 | 20-200 | 0.15-7 | 0.3-3 |
| Li as orotate (mg) | 1-30 | 5-15 | 0.014-0.4 | 0.07-0.2 |

A further preferred ingredient of the composition(s) is a whey concentrate.

The nutrients supplementation composition or combination of compositions according to the invention will mostly be in a form for oral administration, e.g. in oral galenic form or prepared as a composition or combination of compositions ready made for incorporation in a food or feed stuff or beverage.

Oral galenic forms may be liquid, e.g. syrups, or solid, e.g. powders, pills, tablets, optionally coated, granulates, capsules. Where coatings are applied they may be formulated for time release purposes.

The scope of the invention includes such compositions incorporated in a food or feed stuff or beverage, e.g. in the form of a substance selected from the group consisting of maize meal, cassaya meal, baking flour, bread, and mahew (note: mahew is an African slurry-like food, prepared by the lactic acid fermentation of starch, usually from maize or millet meal) enriched with the ingredients defined in the aforegoing.

Also included are compositions as defined above in a form suitable for parenteral administration, e.g. in a form suitable for intravenous injection or perfusion, or intramuscular injection.

The oral application of formulations according to the invention are primarily suitable for prevention of infection with the virus and to prevent the progression of the disease to clinically more severe stages after infection. These preparations generally require some time before effects are seen and they may therefore be less suitable for the treatment of clinically ill patients and especially of the terminally ill.

In these patients, drug treatment may be indicated but it should be remembered that no treatment can be ultimately successful as long as the conditions favourable for virus proliferation exist in the patient. An important reason why virus proliferation proceeds in an uncontrolled manner in these patients are the low blood selenium and glutathione concentrations so typically seen in these patients.

According to the invention, by drastically increasing the blood concentrations of suitable selenium preparations and of glutathione under these conditions, drug (and other) treatments may be rendered more effective and in many cases it may even constitute effective treatment on its own.

However, it is frequently not possible to raise blood levels to the desired levels in the short time usually available before the final demise of the patient. This applies especially in the case of glutathione which cannot be used as such to raise blood levels. The usual precursors (e.g. N-acetylcysteine) for the intracellular production of glutathione react slowly and usually cannot be used to raise glutathione to the desired levels in the limited time available.

The invention therefore also teaches the use of intravenous glutathione to be highly effective in rapidly raising blood glutathione to the desired levels under these conditions.

According to one aspect of the invention, the intravenous administration of glutathione is used to achieve the desired blood levels. For example, in practice, the slow (e.g. 20 minutes) injection of a sterile solution of 400 mg of glutathione dissolved in 20 ml of saline has been found to be effective for this purpose (see examples). Repeated injections may be necessary in severely ill patients.

According to another aspect of the invention, a sterile solution of a suitable biologically available source of selenium (e.g. 1 mg of L-methyl selenocysteine or L-methyl selenomethionine dissolved in 10 ml of sterile saline solution) is used to rapidly raise blood selenium levels to the desired levels (e.g. more than 160 mcg/l) under these conditions. Alternatively, selenium may be administered by the intramuscular route. In this case, a solution for intramuscular administration is prepared by dissolving one or more of the above selenium compounds in an oily or aqueous medium.

According to yet another aspect of the invention, a combination of selenium and glutathione (preferably combined with a gene protector and pH regulator, i.e. an alkalinity enhancer) is used. It is particularly advantageous in the seriously ill AIDS patient, to combine the use of such a preparation with one of the anti-retroviral drugs.

Particularly in the case of galenic forms or where there are reasons to keep certain ingredients apart, it may be preferred to include different ingredients in separate dosage units or formulations for combined use.

A further aspect of the invention comprises the use of the compositions or combinations of compositions as defined and described above for the suppression of viral replication and/or mutation and/or for enhancing the immune system in humans or animals and/or for the prophylaxis or treatment of HIV/AIDS.

Such use may be as part of a regional feeding scheme. It may also be designed to be applied in combination with conventional antiviral and/or anti-retroviral medication.

In the specific context of counteracting serious or potentially serious disease outbreaks or pandemics such as HIV/AIDS, the invention is intended to be applied using strategies combining, where appropriate, broadly applied health care with acute care.

For that reason the invention contemplates a spectrum of regional situations and within this spectrum a differentiation to allow for individual cases.

More specifically, the use of the composition or combination of compositions according to the invention includes the feature that for the control of an HIV/AIDS pandemic in a regional population, such population is stratified into a plurality of different risk groups and different compositions or combinations of compositions are provided in accordance with the different risk magnitudes, within the risk range of low risk=low disease incidence to severely ill HIV-positive persons.

More specifically, the population is stratified into at least the following five risk groups:—
1.) Low risk, low disease incidence group, HIV incidence insignificant.
2.) Normal risk group: HIV status of individuals generally unknown, but on average believed to have average exposure to infection risk.
3.) HIV-positive persons who are still substantially AIDS-symptoms-free and whose CD4 counts are above levels where anti-retroviral drug treatment is indicated.
4.) Clinically ill HIV-positive persons with low CD4 counts in whom symptoms of the AIDS-defining opportunistic infections have already been observed.
5.) Severely ill HIV-positive patients with CD4 counts below 200/mcl.

Firstly, at one end of the spectrum, endangered populations representing risk group 1.) are to be subjected to prophylactic administration of the composition or combination of compositions applying dosage regimens designed to maintaining cost-effectively healthy levels of nutrition in respect of the substances and principles herein described in the greatest number of members of a target population at large:—
(1) in order to maximise overall immunities and minimise infection risks; and
(2) if and when infection (e.g. of HIV) has already occurred, to minimise or delay the development of the infection to a symptomatic diseased condition.

Secondly, risk group 2.), being at the beginning of the intermediate ranges of the spectrum, includes disease free (HIV-negative) persons living in a society where the risk level is judged to be normal on the basis of the level of disease incidence. This group includes a very large percentage of the population as a whole for which nutritional intervention offers the only practical drug based protection, however, one could/should take cognisance of situations, where it becomes apparent that an appreciable percentage of infection has already occurred and individuals can be identified and tested for immune status (CD4 T cell counts), viral counts and possible emergence of symptoms. In that situation, representing group 3.) more potent/higher dosage regimens should be applied, at least to the infected individuals and possibly to the regional population at large. Identified victims should be monitored and treated individually and commensurately with their clinical status, (e.g. depending on whether they are non-symptomatic HIV-positive patients or not). For as long as CD4 counts are above certain levels, retroviral drug treatment would be questionable on the basis of the numbers involved and the real danger of encouraging the emergence of resistant strains of the virus. Appropriate nutritional intervention is especially valuable in this group.

In the case of group 4.), acute treatment is to be applied depending on the severity of symptoms and measurable parameters.

To the extent that abnormalities are observed, mainly restricted to nutritional deficiencies, these are to be treated, applying the principles taught by the present invention, a secondary objective being to maintain the immune status of patients (in terms of CD4 T cell counts) above recognised critical levels. It is currently considered and widely accepted that CD4 T cell counts become critically low at or below 200 CD4 T cells per mm³. At that stage anti-retroviral drug treatment may be indicated in addition to nutritional intervention (advanced formula).

Acute Treatment

It is at present widely accepted amongst HIV/AIDS specialists that conventional anti-retroviral treatment, usually performed with combinations of three drugs including at least one virus inhibitor, a reverse transcriptase inhibitor and at least one proteinase, should be commenced when blood CD4 T cell counts approach or reach the aforesaid critical level of 200, but not before, regardless of viral counts. The present invention teaches adherence to this wisdom, but in addition to apply the above described principles to supplement selenium levels and other nutritional levels, in particular the combination of selenium, GSH and blood alkalinity, preferably involving monitoring those parameters and taking positive corrective action if deficiencies are observed.

According to the invention, the efficacy of such conventional treatment can be enhanced synergistically by the simultaneous administration of the nutritional supplementation regimens taught by the invention. This may in suitable cases allow lower doses of conventional drugs to be used with fewer and less severe side effects.

As regards group 5.), severely ill, HIV-positive patients with CD4 counts below 200/mcl in whom life threatening disease is present, this group must be treated as in group 4.) but in addition may require intravenous glutathione and/or intramuscular selenium and other nutrients.

In applying the above teachings it is important to remember that while each one of the various factors a)-g) discussed has a positive effect in some (but not all) patients, it is when all are simultaneously applied that the maximum effect is achieved. Thus, another novel aspect of the present invention is the recognition that a full effect is only achieved when they are all applied simultaneously. Thus supplementation with selenium alone has some effect in a certain percentage of patients, a much better effect can be expected when the acidosis is corrected which is virtually always present in all AIDS patients, since the enzyme reactions on which the selenium effect is based (glutathione peroxidase) are sensitively dependent on the prevailing blood pH value. Similar synergistic effects exist in the case of the other factors involved.

The present invention further teaches that HIV-positive pregnant mothers and their newborns represent special risk groups within the aforesaid categories for the reasons already stated. Their treatments require special considerations because of the increased risks of rapid development of the disease. Inter alia more extensive and frequent use is to be made here of parenteral modes of supplementation with selenium and other nutrients as taught herein.

In principle, the general teachings of the invention otherwise apply as well. However, control of homocysteine levels is of particular importance, because even without HIV infection, the levels of vitamins controlling homocysteine decline progressively during pregnancy whilst homocysteine levels increase accordingly. These effects are aggravated by HIV infection and must be counteracted, particularly since these effects are also transmitted to the neonates born to such mothers.

The teachings of the invention relating to anti-inflammatory treatment are of particular importance in the treatment of HIV-positive pregnant women and their neonates in order to block or reduce the pathway for viral transmission through cell walls.

Likewise, the control of acidity as taught by the invention is of even greater importance in the treatment of HIV-positive women and their neonates than in the case of other HIV-positive patients.

In the context of HIV/AIDS the invention has particular significance in its effect on the action of reverse transcriptase.

The HIV as well as other retroviruses contain their genetic information in the form of RNA stored inside a protein and lipid icosahedral shell. This spherical virus particle is further surrounded by an envelope consisting of virus specific encoded glycoprotein molecules in a lipid bilayer derived from the plasma membrane of the host cell. The virus penetrates the host cell by fusion of the proteins in the viral envelope through interaction with a specific plasma membrane receptor situated on the surface of the host cell (e.g. the CD4 immune cell). Once inside the host cell, new and atypical DNA molecules are synthesised inside the host cell nucleus by an enzyme initially called RNA-directed DNA polymerase. This enzyme was later called reverse transcriptase. Thus the integration of the viral RNA with the host cell nucleus is part of the life cycle of the HIV.

It is this vital step that is suppressed by antimutagenic agents such as chlorophyllin. The RNA genome of HIV contains several genes and also encodes several proteins all of which stimulate transcription and translation.

The presence of a multiplicity of these stimulatory factors and rapid variation of the viral envelope protein which allow the rapid development of mutant strains of the virus, is one of the reasons why it is so difficult to produce either an effective vaccine or drug to combat the disease. Whilst these efforts must continue, the present invention therefore postulates that a more immediate solution to the HIV/AIDS problem should in addition be sought in strengthening the host immune system and its built-in glutathione-based anti-virus mechanisms.

The HIV virus belongs to the group of retroviruses which carry their genetic information encoded in RNA in contrast to human cells where the genetic code is carried in the cell nuclei encoded in DNA. Before the HIV virus can replicate and multiply in human cells, it has to transcribe its genetic code from RNA to DNA “language”. This process is known as reverse transcription. Reverse transcription can be seen as a process of viral induced gene mutation. In order for reverse transcription to occur, the virus must come into close contact with the DNA in the host's cells. A similar association occurs when environmental and other carcinogens penetrate the genetic material in human cells to cause cancer by inducing mutations. The first line of defence against many chemical carcinogens is agents that prevent gene mutation. Many of these agents occur in natural products including a chlorophyll derivative known as chlorophyllin.

Chlorophyllin is a modified, water soluble form of chlorophyll that has been used as an anti-mutagenic substance in cancer studies for many years. There is therefore a large body of data dealing with the anti-cancer and anti-mutagenic effects of chlorophyllin(see for example Environm Mol Mutagen 1997, 30: 468). No prior art studies have been done on the effects of chlorophyllin on clinical AIDS.

A pivotal study, published in 1986, showed that chlorophyllin is a more effective anti-mutagenic compound than all the other known anti-cancer compounds at that time (Mutation Research 1986, 173:111). This study therefore demonstrated the extra-ordinary effectiveness of chlorophyllin to inhibit deadly gene mutations caused by chemical compounds.

Surprisingly, it has now been found that chlorophyllin and other anti-mutagenic substances effective in cancer therapy also inhibit the mutation of retroviruses and viral growth, although there was no reason to suspect this. Indeed, from further studies and considerations it becomes apparent that the anti-mutagenic and viral growth suppressing effects of chlorophyllin are exerted through two different mechanisms. Firstly, chlorophyllin reacts with chemical carcinogens such as certain heterocyclic amines by chemically forming adducts with the carcinogens thus preventing them from reacting with the nuclear DNA material (Cancer Letters 1996, 107:223). However, chlorophyllin also associates with DNA in the genetic material thus preventing it from being damaged by mutagenic agents. The invention teaches that chlorophyllin protects the DNA in normal cells against HIV attack by means of a similar mechanism.

Indole-3-carbinol (I3C) is a further naturally occurring DNA protector and anti-mutagenic agent that has been shown to prevent up to 90% of chemically induced cancers. Further studies have shown that I3C decreases the DNA damage in various tissues by 67-82%. Its principal mode of action is to prevent the various carcinogens from forming adducts with DNA. (Food Chem Toxicol 2000, 38:15). According to the invention, I3C protects the DNA in normal cells against attack by HIV by means of a similar mechanism.

Folic acid supplements have been shown in several studies to prevent cancer in humans, especially colon and breast cancer. This can also be traced to gene protection as in the case of the other two compounds discussed above.

Niacin (niacinamide) (10) has been consistently reported to be severely depleted in AIDS patients (9). Several studies have demonstrated the fact that niacinamide may protect DNA in cells from damage. These studies have been limited to the protective effect of niacin against aging of brain cells and beneficial effects of niacinamide supplementation in AIDS patients (8). However, none of these studies has investigated the protective effect of niacin or niacinamide against virus-induced DNA damage and specifically against DNA damage associated with the HIV.

Deficiencies in cysteine, glutamine and tryptophan also contribute to the major clinical symptoms of AIDS (Townsend Letter, April 2002, p76). Optimal treatment of the clinically ill AIDS patient must therefore include not only selenium in a biologically available form, but also these critical amino acids. The applicant and others have observed that tryptophan deficiencies in AIDS patients result in pellagra like symptoms, including depression.

The applicant is aware that selenium absorption may be a problem, at least in some patients with special reference to AIDS patients. In these patients it is therefore appropriate to devote special attention to the amount and type of selenium compounds used in supplementation programs. In addition, applicant has found that compounds that improve the condition of the cells in the gut lining (e.g. glutamine) as well as substances that improve gastrointestinal absorption (e.g. piperine) may be used to improve selenium absorption.

The invention is aimed at addressing at least some of the following conditions or objectives:
1) an optimally functional glutathione system;
2) the presence of natural compounds that prevent or retard the association of the virus with the DNA of the host thus protecting the host's genetic material against viral attack;
3) Supplying sufficient quantities of the other nutrients that are also encoded by the virus and of which critical shortages may therefore develop in the AIDS patient;
4) Ensuring adequate blood levels of selenium by selenium supplementation utilising different biologically available selenium compounds and by introducing steps to normalise selenium absorption in those patients in which this is a problem;
5) AIDS is a catabolic disease in which widespread cachexia with loss of muscle and tissue occurs. Limiting these losses and possibly replacing lost tissue must therefore be a prime therapeutic objective. Apart from the usual measures to achieve this (improved nutrition, high quality protein, general multi-mineral and vitamin supplementation), the applicant has surprisingly found that loss of sulphur is an important aspect of tissue loss; and/or
6) Loss of sulphur is particularly serious in the AIDS patient since this implies reduced biosynthesis of sulphur amino acids cysteine and methionine, both of which are of importance in the AIDS patient.

The invention is therefore aimed at controlling the HIV/AIDS pandemic in the masses of people (especially young people) in Third World countries where the use of expensive drugs and critical institutionalised care are excluded due to economic and logistical factors. In some variations of the application, the product is formulated in such a way that, apart from inhibiting replication of the AIDS virus, the product also serves as nutritional supplement, which addresses some of the many deficiencies that occur in the target population. In order to reach as wide a segment of the target population as possible and for economic reasons, the invention provides for different formulations of the invention to be used in different segments of the population.

### DETAILED DESCRIPTION

The invention is now described by way of non limiting examples.

A) For the Prevention of AIDS in the Population at Large

It will be appreciated that such a formula must be cost effective. The product will be administered to those at risk in the form of suitably formulated pills or tablets to be taken on a daily basis. The product can also be administered by enriching basic food items such as bread, maize flour, soy flour or any other suitable food item by means of methods and procedures known in the art.

### EXAMPLE 1

### Per Daily Dose:

SELENIUM:

| | |
|---|---|
| Selenium (as 0.1% yeast complex) | 0.20 mg |
| Total elemental selenium | 0.20 mg |

GLUTATHIONE SOURCE:

| | |
|---|---|
| N-acetylcysteine | 200 mg |
| Riboflavin | 5 mg |
| Niacinamide | 20 mg |
| Folic acid | 1.0 mg |
| Trace mineral source | 200 mg |

ACIDITY CONTROL SYSTEM:

| | |
|---|---|
| Calcium carbonate | 500 mg |
| Magnesium carbonate | 400 mg |

Tablets are made in the usual manner according to procedures well-known in the art

DIRECTIONS FOR USE: One to two tablets daily on an empty stomach.

### EXAMPLE 2

For the Prevention of AIDS in Populations (Simplified Formula)

| | | |
|---|---|---|
| Selenium (as 0.1% yeast complex) | 200 | μg |
| Niacinamide | 20 | mg |
| Folic acid | 1 | mg |
| Calcium carbonate | 400 | mg |
| Trace mineral source | 100 | mg |

### EXAMPLE 3

For the Prevention of AIDS in Populations (Fortified Formula)

SELENIUM:

| | |
|---|---|
| Selenium (as 0.1% yeast complex) | 0.3 mg |
| Selenium (as amino acid complex) | 0.1 mg |
| Total selenium | 0.4 mg |

GLUTATHIONE:

| | |
|---|---|
| L-cysteine | 400 mg |
| MSM | 200 mg |
| Riboflavin | 5 mg |
| Niacinamide | 20 mg |

ANTI-MUTAGENICS

| | |
|---|---|
| Folic acid | 1.0 mg |

ACIDITY CONTROL SYSTEM:

| | |
|---|---|
| Calcium carbonate | 500 mg |
| Basic magnesium carbonate | 400 mg |

TRACE MINERALS

| | |
|---|---|
| Sea water extract (or purified soil trace elements) | 300 mg |

B) For the Treatment of Asymptomatic Patients that Have Been Exposed to the HIV Virus (Non-Symptomatic HIV Positive Patients)

### EXAMPLE 4

### Per Daily Dose:

SELENIUM:

| | | |
|---|---|---|
| Selenium (as 0.2% amino acid complex) | 0.2 | mg |
| Selenium (as 0.1% selenium yeast) | 0.2 | mg |
| Total selenium | 0.4 | mg |

GLUTATHIONE SOURCE:

| | | |
|---|---|---|
| N-Acetylcysteine | 300 | mg |
| L-cysteine | 500 | mg |
| Riboflavin | 5 | mg |
| Niacinamide | 20 | mg |
| MSM | 200 | mg |

GENE PROTECTORS (anti mutagenic compounds):

| | | |
|---|---|---|
| Folic acid | 5.0 | mg |
| Chlorophyllin | 300 | mg |

ANTI-INFLAMMATORIES

| | | |
|---|---|---|
| Quercitin | 300 | mg |

MINERALS:

| | | |
|---|---|---|
| Calcium carbonate | 500 | mg |
| Basic magnesium carbonate | 200 | mg |

TRACE MINERALS

| | | |
|---|---|---|
| Sea water extract | 300 | mg |

VITAMINS:

| | | |
|---|---|---|
| Pyridoxine (as pyridoxine HCl) | 5 | mg |
| Vitamin E (as d-α-tocopherol acetate) | 20 | mg TE |

PROBIOTIC

| | | |
|---|---|---|
| Mix of probiotic intestinal microorganisms | 500 | mg |

### EXAMPLE 5

For the Preventative Treatment of HIV Positive Patients (Fortified Formula)

### Per Daily Dose:

SELENIUM:

| | | |
|---|---|---|
| Selenium (as 0.2% amino acid complex) | 0.1 | mg |
| Selenium (as selenium yeast) | 0.3 | mg |
| Total selenium | 0.4 | mg |

GLUTATHIONE SOURCE:

| | | |
|---|---|---|
| L-cysteine | 400 | mg |
| Riboflavin | 5 | mg |
| Niacin | 20 | mg |
| MSM | 300 | mg |

DNA PROTECTORS (anti-mutagenic compounds):

| | | |
|---|---|---|
| Chlorophyllin | 300 | mg |
| Folic acid | 5 | mg |

HOMOCYSTEINE CONTROL

| | | |
|---|---|---|
| Betaine | 300 | mg |
| ANTI-INFLAMMATORIES | | |
| Quercitin | 500 | mg |

MINERALS: (ACIDITY CONTROL)

| | | |
|---|---|---|
| Potassium citrate | 400 | mg |
| Calcium carbonate | 500 | mg |
| Magnesium (as oxide) | 200 | mg |

TRACE MINERAL SOURCE

| | | |
|---|---|---|
| Zinc oxide | 30 | mg |
| Manganese (as citrate or sulphate) | 5 | mg |

VITAMINS:

| | | |
|---|---|---|
| Pyridoxine (as hydrochloride) | 5 | mg |
| d-α-tocopherol acetate (Vitamin E) | 20 | mg TE |

PROBIOTICS

| | | |
|---|---|---|
| Lyophilised mix of intestinal probiotic organisms | 500 | mg |

DIRECTIONS FOR USE: 6-10 tablets daily in divided doses on an empty stomach

The product can be administered in the form of tablets, capsules, capslets, a powder, a specially prepared drink or food or any other suitable vehicle.

C) For the Treatment of Clinical AIDS

### EXAMPLE 6

### Per Daily Dose:

SELENIUM:

| | | |
|---|---|---|
| Selenium (as selenomethionine) | 0.26 | mg (=0.1 mg Se) |
| Selenium (as 0.1% selenium yeast) | 100 | mg (=0.1 mg Se) |
| Selenium (as amino acid chelate, 0.2% | 100 | mg (=0.2 mg Se) |
| Selenium (complexed as coral calcium) | 500 | mg (=0.01 mg Se) |
| Total selenium | 0.41 | mg |
| Lithium as orotate | 5 | mg |

GLUTATHIONE SOURCE:

| | | |
|---|---|---|
| α-lipoic acid | 400 | mg |
| N-acetylcysteine | 600 | mg |
| L-cysteine | 400 | mg |
| Riboflavin | 5 | mg |
| MSM | 500 | mg |
| Niacinamide | 40 | mg |

GENE PROTECTORS (anti-mutagenic compounds):

| | | |
|---|---|---|
| Chlorophyllin | 300 | mg |
| Indole-3-carbinol (I-3-C) | 500 | mg |
| Folic acid | 5 | mg |

HOMOCYSTEINE CONTROL

| | | |
|---|---|---|
| Betaine (trimethylglycine) | 300 | mg |

ALKALINITY ENHANCER:

| | | |
|---|---|---|
| Calcium (200 mg) as coral calcium | 1000 | mg |
| Basic magnesium carbonate | 300 | mg |
| Potassium citrate | 400 | mg |

GASTROINTESTINAL PROTECTOR:

| | | |
|---|---|---|
| L-Glutamine | 1000 | mg |
| Lyophilised probiotic culture mix | 300 | mg |

CYTOKINE CONTROL SYSTEM

| | | |
|---|---|---|
| Nettle leaf extract 1% | 500 | mg |
| Quercitin | 1000 | mg |

ADDITIONAL NUTRIENTS AND ANTIOXIDANTS

| | | |
|---|---|---|
| Zinc oxide (=30 mg zinc) | 37.34 | mg |
| Pyridoxine HCl | 12.2 | mg |
| d-α-tocopherol acetate | 30 | mg |
| Ascorbic acid | 200 | mg |

DOSAGE: 10-12 tablets daily in divided doses on an empty stomach
NOTE: The formula is suitable for use in conjunction with drug treatment of AIDS patients and may be expected to potentiate the effects of most of these drugs.

### EXAMPLE 7

CORRECTION OF CRITICAL NUTRIENT DEFICIENCIES IN
AIDS PATIENTS (excluding selenium; to be administered
in addition to selenium formulation)

| | | |
|---|---|---|
| L-Tryptophan | 500 | mg |
| L-cysteine | 500 | mg |
| L-glutamine | 500 | mg |
| Zinc (as amino acid chelate) | 30 | mg |

VITAMINS:

| | | |
|---|---|---|
| Vitamin B1(thiamine) | 10 | mg |
| Pyridoxine (vitamin B6) | 5 | mg |
| Vitamin E (as d-α-tocopherol acetate) | 30 | TE |
| Niacin | 50 | mg |
| Vitamin A | 4000 | mcg RE |
| β-Carotene | 30 | mg |
| Ca-d-pantothenate | 20 | mg |
| Ascorbic acid | 500 | mg |
| Vitamin B12 | 100 | mcg |
| Folic acid | 5 | mg |
| Riboflavin | 5 | mg |
| Biotin | 0.2 | mg |
| Manganese (as citrate or sulphate) | 5.0 | mg |
| Vitamin C | 300 | mg |
| Magnesium (as oxide) | 100 | mg |

DIRECTIONS FOR USE: 4-6 Tablets daily in divided doses on an empty stomach daily.

The product can be administered in the form of tablets, capsules, capslets, as powder, or incorporated in suitable food items or as a special drink.

The Administration of Products According to the Invention to Whole Populations

The control of the HIV/AIDS pandemic calls for the treatment of entire populations. This for, obvious reasons, is impossible with conventional drugs. Educational and sociological approaches have also largely failed in the past, mainly because these depend on the active cooperation of individuals in the population at risk. The remaining alternative is to administer anti-AIDS formulations to suitable carriers such as food items, drinking water, specially formulated drinks etc.

The present invention is particularly suitable for such purposes for the following reasons:
the product according to the invention is largely tasteless.
the effects of the product are only seen after long term administration (weeks to months) making it particularly suitable for this type of administration.
relatively small quantities daily are required
the product is stable
the product is non-toxic, even when consumed by children over long periods
the product has other health advantages apart from acting to suppress the proliferation of the HIV virus. For example, the selenium in the formulation acts as powerful anti-oxidant which inter alia protects the immune system thus increasing the resistance to the many other infections to which the target populations are subjected to.

The most obvious vehicle to administer the product is by the addition of the product to some basic food item such as maize flour which is consumed by everyone in the target population.

The Enrichment of Maize Flour with Products According to the Invention

The following non-limiting example illustrates the use of one formulation of the invention in the enrichment of maize flour:

Maize Flour Additives (MFA) According to the Invention

### EXAMPLE 8

### Maize Flour Additive(Low Level) (MFA)

NOTE: This product is intended for low risk populations

The following ingredients are intimately mixed to prepare the additive:

| Additive | Daily dose | | % | 1 kg additive | |
|---|---|---|---|---|---|
| Selenium 0.1% AAC (=200 μg Se) | 200 | mg | 33.3 | 333 | g |
| Folic acid | 2.0 | mg | 0.33 | 3.3 | g |
| Niacinamide | 10 | mg | 1.67 | 16.7 | g |
| Riboflavin | 2 | mg | 0.33 | 3.3 | g |
| NAC | 50 | mg | 8.33 | 83.3 | g |
| Calcium carb (=108 mg Ca) | 270 | mg | 45 | 450 | g |
| Mag oxide (=40 mg Mg) | 66 | mg | 11 | 110 | g |
| | 600 | mg | | 1000 | g |

To prepare enriched maize flour, add 200 g of MFA to 100 kg of maize flour.

### EXAMPLE 9

### Maize Flour Additive (High Level) (MFA-H)

NOTE: This product is intended for high risk populations

| | | | | 1 kg | |
|---|---|---|---|---|---|
| Additive | Daily dose | | % | additive | |
| Selenium AAC 0.1% (=400 μg Se) | 0.4 | mg | 0.032 | 0.32 | g |
| L-cysteine | 400 | mg | 16.01 | 160 | g |
| Riboflavin | 5 | mg | 0.5 | 4.0 | g |
| Folic acid | 2 | mg | 0.16 | 1.6 | g |
| Calcium carb (=500 mg Ca) | 750 | mg | 60.05 | 600.5 | g |
| Mag oxide (=100 mg Mg) | 166 | mg | 13.29 | 132.9 | g |
| Pyridoxine.HCI (=5 mg vitamin B6) | 6.1 | mg | 0.49 | 4.9 | g |
| Niacinamide | 20 | mg | 1.6 | 16 | g |
| NAC | 100 | mg | 8.06 | 80.6 | g |
| | 1449.5 | | | 1000 | g |

To prepare enriched maize meal, mix 417 g of the additive intimately into 100 kg of maize flour.

### EXAMPLE 10

Multivitamin/Mineral Supplement for AIDS Patients

| | ACTIVES | | | | |
|---|---|---|---|---|---|
| | PER DAILY | | | QTY | |
| INGREDIENT | DOSE: | | MATERIAL | MATERIAL | |
| Selenium | 0.4 | mg | 0.2% amino acid chelate | 200 | mg |
| Folic acid | 1.0 | mg | folic acid | 1.0 | mg |
| Niacinamide | 30 | mg | niacinamide | 30 | mg |
| L-glutamine | 1000 | mg | L-glutamine | 1000 | mg |
| Vitamin A | 3000 | IU | Type500CWS 500 IU/mg | 6 | mg |
| Thiamine | 2 | mg | Thiamine.HCL | 2.3 | mg |
| Riboflavin | 2 | mg | Riboflavin | 2 | mg |
| Pyridoxine | 5 | mg | Pyridoxine.HCl | 6.1 | mg |
| Vitamin C | 100 | mg | Type EC coated Roche | 111.1 | mg |
| Vitamin E | 20 | RE | d-α-tocopherol acetate | 23.1 | mg |
| Magnesium | 100 | mg | Mag oxide | 164 | mg |
| Calcium | 300 | mg | Calcium carb | 750 | mg |
| Zinc | 20 | mg | Zinc oxide | 25 | mg |
| Pantothenic acid | 5 | mg | Ca-d-pantothenate | 5.4 | mg |
| Copper | 1 | mg | Copper sulphate.5H2O | 3.93 | mg |

Dosage: 2 tabs twice daily with meals.

### EXAMPLE 11

Soy Based Fortified Protein Drink for AIDS Patients

| | | | | QTY | |
|---|---|---|---|---|---|
| INGREDIENT | ACTIVES | | MATERIAL | MATERIAL | |
| SOY | SOY | | SOY MILK | 10 | g |
| | FLOUR | | POWDER | | |
| Selenium | 200 | μg | selenium AAC 0.2% | 0.1 | g |
| Folic acid | 1 | mg | folic acid | 1 | mg |
| L-cysteine | 100 | mg | L-Cysteine | 100 | mg |
| Calcium | 100 | mg | Calcium carb | 250 | mg |
| Magnesium | 50 | mg | Mag oxide | 83 | mg |
| Pyridoxine | 2 | mg | pyridoxine.HCl | 2.4 | mg |

Dosage: Mix 2-3 teaspoonfuls in a glass of milk, juice or water.
Further Examples

The following non-limiting examples illustrate the application of the invention in different population groups and different clinical situations.

### EXAMPLE 12

Parenteral Administration of Products According to the Invention

A. A Solution Containing the Following Ingredients per Litre of Physiological Saline is Prepared for Intravenous Administration:

| | Preferred | Range | |
|---|---|---|---|
| L-methyl | 40.5 mg (=10 mg of selenium) | 5-200 | mg |
| selenocysteine | | | |
| L-glutathione | 20.0 mg | 10-500 | mg |
| Riboflavin | 100 mg | 10-1000 | mg |
| Niacinamide | 250 mg | 20-1000 | mg |
| Folio acid | 100 mg | 10-500 | mg |
| Potassium bicarbonate | 500 mg | 100-2000 | mg |
| α-lipoic acid | 400 mg | 100-800 | mg |

After dissolving the ingredients in the solvent, the solution is adjusted to 7,4 and the solution is then sterilised by means of filtration according to technology known in the art.

After filtration, the sterile solution is dispensed in dark coloured 20 ml vials or other suitable dark coloured containers for sterile liquids and stored at 4 degrees Celsius. This solution contains (per 20 ml vial) the following active ingredients:

| | |
|---|---|
| L-methyl selenocysteine | 0.81 mg (200 mg Se) |
| L-glutathione | 0.4 mg |
| Riboflavin | 2.0 mg |
| Niacinamide | 5.0 mg |
| Folic acid | 2.0 mg |
| Potassium bicarbonate | 10 mg |
| α-lipoic acid | 8 mg |

In order to administer, one vial (20 ml) is diluted in 20 ml sterile saline solution and the mixture injected slowly intravenously over a 20-25 min. period of time. In the beginning, 1-2 vials are administered in this manner 3 times a week for 2 weeks. Thereafter, and depending on the condition of the patient, dosage frequency may be reduced to 1-2 weekly. The preparation should only be administered by a medical practitioner.

B. A Solution Containing the Following Ingredients per 200 ml of Physiological Saline is Prepared for Intramuscular Administration of the Product:

| | Example | Range | |
|---|---|---|---|
| L-methyl | 40.5 mg (=10 mg of selenium) | 5-200 | mg |
| selenocysteine | | | |
| L-glutathione | 20.0 mg | 10-500 | mg |
| Riboflavin | 100 mg | 10-1000 | mg |
| Niacinamide | 250 mg | 20-1000 | mg |
| Folic acid | 100 mg | 10-500 | mg |
| Potassium bicarbonate | 500 mg | 100-2000 | mg |
| α-lipoic acid | 400 mg | 100-800 | mg |

After dissolving the ingredients in the solvent, the pH of the solution is adjusted to 7,4 and the solution is then sterilised by means of filtration according to technology known in the art.

After filtration, the sterile solution is dispensed in dark coloured 5 ml vials or other 15 suitable dark coloured containers for sterile liquids and stored at 4 degrees Celsius.

This solution contains (per 5 ml vial) the following active ingredients:

| | |
|---|---|
| L-methyl selenocysteine | 0.81 mg (200 mcg Se) |
| L-glutathione | 0.4 mg |
| Riboflavin | 2.0 mg |
| Niacinamide | 5.0 mg |
| Folic acid | 2.0 mg |
| Potassium bicarbonate | 10 mg |
| α-lipoic acid | 8 mg |

In order to administer, one vial (5 ml) is injected by deep intramuscular injection. In the beginning, 1-2 vials are administered in this manner 3 times a week for 2 weeks. Thereafter, and depending on the condition of the patient, dosage frequency may be reduced to 1-2 weekly. The preparation should only be administered by a medical practitioner.

### EXAMPLE 13

Selenium Formula for the Treatment of HIV Infected Pregnant Women: Second Trimester and After

### Per Daily Dose:

| SELENIUM: | | |
|---|---|---|
| Selenium (as 0.2% amino acid complex) | 0.1 | mg |
| Selenium (as 0.1% yeast complex) | 0.2 | mg |
| Selenium (as selenomethionine) | 0.1 | mg |
| Total selenium | 0.4 | mg |
| GLUTATHIONE SOURCE: | | |
| L-Cysteine | 400 | mg |
| MSM (methyl sulfonylmethane) | 400 | mg |
| Riboflavin | 5 | mg |
| Niacinamide | 50 | mg |
| ANTI-MUTAGENIC: | | |
| Folic acid | 5.0 | mg |
| ACIDITY CONTROL SYSTEM: | | |
| Potassium citrate | 400 | mg |
| Calcium carbonate | 500 | mg |
| Basic magnesium carbonate | 500 | mg |
| FOR CONTROL OF HOMOCYSTEINE LEVELS: | | |
| Trimethylglycine | 500 | mg |
| | 2760.8 | mg |

Tablets or capsules or capslets are made according to procedures well known in the art.

DIRECTIONS FOR USE: 4-5 tablets daily in divided doses on an empty stomach.

### EXAMPLE 14

Selenium Formula for the Treatment of Neonates Born to HIV Positive Mothers

### Daily Dose per kg Body Weight

| SELENIUM: | | |
|---|---|---|
| Selenium (as methyl selenocysteine) | 5.0 | mcg |
| GLUTATHIONE SOURCE: | | |
| N-Acetylcysteine | 3.0 | mg |
| Riboflavin | 0.07 | mg |
| Niacinamide | 0.3 | mg |
| α-lipoic acid | 25 | mg |
| ANTIMUTAGENIC: | | |
| Folic acid | 0.05 | mg |
| TO CONTROL HOMOCYSTEINE LEVELS: | | |
| Vit B6 | 0.05 | mg |
| Vit B12 | 3.0 | mcg |
| Trimethylglycine (betaine) | 10 | mg |
| GLUCOSE: | 50 | mg |
| TABLET DISINTEGRANT | 50 | mg |

Rapidly disintegrating tablets containing the above quantities per tablet are made according to procedures well known in the art.

DIRECTIONS FOR USE: Dissolve the required number of tablets (determined by body weight) in collected mother's milk or infant formula in such a manner that the total daily dose is administered in no less than 3 feeds. Thus an infant weighing 5 kg would require a total of 5 tablets dissolved in milk daily. This dose should then be administered in 5 doses throughout the day using 1 tablet per feed.

### EXAMPLE 15

Multivitamin/Mineral Formulation for HIV Positive Pregnant Women (to be Combined with Example 12)

### Per Daily Dose:

Components often Deficient in AIDS Patients:

COMPONENTS OFTEN DEFICIENT IN AIDS PATIENTS:

| | | |
|---|---|---|
| Vitamin A | 1500 | mcg RE |
| β-Carotene | 20 | mg |
| Pyridoxine | 25 | mg |
| Ascorbic acid | 500 | mg |
| Vitamin E | 50 | mg TE |
| Vitamin B12 | 50 | mcg |
| Folic acid | 5 | mg |
| α-lipoic acid | 300 | mg |
| Quercitin | 500 | mg |
| Magnesium (as oxide) | 200 | mg |
| Zinc (as oxide) | 50 | mg |

ADDITIONAL SYNERGISTIC COMPONENTS:

| | | |
|---|---|---|
| Thiamine (as chloride) | 10 | mg |
| Riboflavin | 5 | mg |
| Niacin | 30 | mg |
| Ca-d-pantothenate | 20 | mg |
| Biotin | 0.2 | mg |

### EXAMPLE 16

Rapidly Disintegrating Multivitamin/Mineral Formula for Infants:

### Per kg Body Weight per Day:

VITAMINS THAT ARE FREQUENTLY DEFICIENT IN HIV
POSITIVE INFANT

| | | |
|---|---|---|
| Vitamin A | 20 | mcg RE |
| β-Carotene | 0.2 | mg |
| Pyridoxine | 0.3 | mg |
| Ascorbic acid | 10 | mg |
| Vitamin E | 0.7 | mg TE |
| Vitamin B12 | 0.7 | mcg |
| Folic acid | 0.07 | mg |
| α-lipoic acid | 2.0 | mg |
| Quercitin | 5.0 | mg |
| Inositol | 1.0 | mg |
| L-Carnitine | 5.0 | mg |
| Magnesium (as chelate) | 3 | mg |
| Zinc (as chelate) | 0.7 | mg |

SYNERGISTIC COMPOUNDS:

| | | |
|---|---|---|
| Thiamine | 0.1 | mg |
| Riboflavin | 0.07 | mg |
| Niacinamide | 0.3 | mg |
| Ca-d-pantothenate | 0.2 | mg |
| Biotin | 0.002 | mg |

### EXAMPLE 17

Nutritional Supplement for HIV+ Women

### Per Daily Dose:

| Whey protein concentrate | 40 | g (=28 g prot.) |
|---|---|---|
| Soya milk powder | 100 | g (=12 g prot.) |
| Sorghum rice powder | 100 | g |
| Sweetener | q.s. | |
| Flavouring | q.s. | |
| | 240 | g |

The claims which follow are to be considered an integral part of the present disclosure. The term “comprises” or “comprising” as used herein and in the claims, has its customary non-restrictive meaning which denotes that in addition to any items to which the term relates, there may be included additional items not specifically mentioned.

What is claimed is:

1. A nutrient supplementation composition or combination of compositions comprising
a) one or more biologically absorbable and acceptable selenium compounds in synergistic combination with substances enhancing physiological selenium absorption and utilisation and further including any one of the following features:
(1) that a) is represented, in amounts to provide a daily dosage of not less than about 400 mcg Se, by
a1) at least one selenium compound selected from methyl selenocysteine, selenomethionine, selenium yeast complex, selenium amino acid complex and/or
a2) a plurality of selenium compounds;
(2) that there is present
c) a combination of biologically absorbable and acceptable blood and intracellular alkalinity enhancing components in amounts for enhancing blood pH to above 7,45, including
c1) one or more salts of calcium and/or magnesium and/or
c2) an alkalinity enhancing potassium compound as represented by a lactate, tartrate, malate or other fruit acid salt, or bicarbonate, analogues and derivatives of the aforegoing and combinations of a plurality of the aforegoing. and
c3) in combination with a source of calcium one or more salts of cesium and/or rubidium and/or
c4) a lithium salt.
(3) a combination of (1) and (2)

2. A nutrient supplementation composition or combination of compositions as claimed in claim 1 comprising b) one or more biologically absorbable and acceptable sources of or precursors of glutathione (GSH) and a combination of (1) and (2).

3. The composition or group of compositions as claimed in claim 1 comprising in addition the following combination of features:—
d) a biologically absorbable and acceptable source of sulphur; and
e) one or more biologically absorbable and acceptable anti-mutagenic compounds; and
f) in the event of compositions for oral administration, one or more gastrointestinal absorption enhancers for selenium; and
g) one or more gastrointestinal protectors.

4. The composition or combination of compositions as claimed in claim 1, wherein one or more anti-mutagenic compounds is/are present including at least chlorophyllin in an amount suitable to provide a daily dosage level of about 50-1000 mg and/or indole-3-carbinol in an amount suitable to provide a daily dosage level of about 50-1000 mg.

5. The composition or combination of compositions as claimed in any one of claims 1 in a form for oral administration, selected from the group consisting of compositions in oral galenic form; compositions ready made for incorporation in a food or feed stuff or beverage; compositions incorporated in a food or feed stuff or beverage; compositions in the form of a substance selected from the group consisting of maize meal, cassaya meal, baking flour, bread, and mahew (note: mahew is an African slurry-like food, prepared by the lactic acid fermentation of starch, usually from maize or millet meal), enriched with the ingredients as defined; and combinations of the aforegoing.

6. The composition or combination of compositions as claimed in claim 1 in a form selected from the group consisting of compositions suitable for parenteral administration; compositions in a form suitable for intravenous injection or perfusion; compositions, including glutathione in a form for intravenous administration; and combinations of the aforegoing.

7. The composition or combination of compositions according to claim 1, which includes a substance selected from the group consisting of a source of lithium; a source of lithium selected from organic lithium salts of the group consisting of lithium orotate, lithium aspartate, lithium salts of fatty acids, polyunsaturated fatty acids, those that occur in phospholipids in cellular membranes; DHA, EPA, x-linolenic acid, palmitic acid, stearic acid and other acids that occur in biological membranes or from lithium selenite or lithium selenate and combinations of a plurality of the aforegoing.

8. The composition or combination of compositions as claimed in claim 7, which contains the source of lithium in “ultra low” dosage units of 20-500 mcg lithium.

9. The composition or combination of compositions as claimed in claim 1, which contains humic acid and/or fulvic acid and/or bioflavonoids and/or GSH, analogues and derivatives of the aforegoing and combinations of a plurality of the aforegoing.

10. The composition or combination of compositions as claimed in claim 1, which contains one or more substances for modulating cytokine activity, selected from the group consisting of substances suppressing Tumor Necrosis Factor-alpha (TNFα) and interleukins 1 and 6, nettle leaf extract, pentoxifilline, curcumine, antioxidants, NAC, α-lipoic acid, analogues and derivatives of the aforegoing and combinations of a plurality of the aforegoing.

11. The composition or combination of compositions as claimed in claim 1, which includes a substance or combination of substances for reducing homocysteine levels in blood.

12. The composition or combination of compositions as claimed in claim 1, which is for a use selected from the group consisting of suppression of viral replication and/or viral mutation in humans or animals; the prophylaxis or treatment of HIV/AIDS.

13. The composition or combination of compositions as claimed in claim 12, including a source of probiotics.

14. A use of a composition or combination of compositions as claimed in claim 1 for the manufacture of a medicament for the suppression of viral replication and/or mutation and/or for enhancing the immune system in humans or animals; and/or for the manufacture of a medicament for the prophylaxis or treatment of HIV/AIDS; and/or for the treatment of HIV-positive pregnant women in order to reduce their HI-viral loads, and/or strengthen their immune system and/or mitigate or delay the onset of AIDS symptoms and/or reduce the risk of and/or counteract the effect on the foetus and neonate of mother-to-child transmission (MTCT) prior to, during or after parturition and/or to the treatment of newborn infants of such women.

15. The use as claimed in claim 14, wherein for the control of an HIV/AIDS pandemic in a regional population, such population is stratified into a plurality of different risk groups and different compositions or combinations of compositions are provided in accordance with the different risk magnitudes, within the risk range of low risk=low disease incidence to severely ill HIV-positive persons, and more particularly in that the population is stratified into at least the following five risk groups:—
1.) Low risk, low disease incidence group, HIV incidence insignificant.
2.) Normal risk group: HIV status of individuals generally unknown, but on average believed to have average exposure to infection risk.
3.) HIV-positive persons who are still substantially AIDS-symptoms-free and whose CD4 counts are above levels where anti-retroviral drug treatment is indicated.
4.) Clinically ill HIV-positive persons with low CD4 counts in whom symptoms of the AIDS-defining opportunistic infections have already been observed.
5.) Severely ill HIV-positive patients with CD4 counts below 200/mcl.
